(19) United States
(12) Patent Application Publication
(10) Pub. No.: US 2024/0216908 A1
(43) Pub. Date: Jul. 4, 2024

(54) LATERAL FLOW ANALYTICAL DEVICE AND METHOD

(71) Applicant: IDEXX Laboratories, Inc., Westbrook, ME (US)

(72) Inventors: Murthy Yerramilli, Westbrook, ME (US); Eugene Chan, Westbrook, ME (US); William Foster, Westbrook, ME (US); Jennifer Gagner, Westbrook, ME (US)

(21) Appl. No.: 18/390,889

(22) Filed: Dec. 20, 2023

Related U.S. Application Data
(60) Provisional application No. 63/436,280, filed on Dec. 30, 2022.

(51) Int. Cl. B01L 3/00; G01N 33/543

(52) U.S. Cl. CPC B01L 3/5023; G01N 33/54388; B01L 2200/0689; B01L 2200/12; B01L 2200/16; B01L 2300/0672; B01L 2300/0681; B01L 2300/069; B01L 2300/12

(57) ABSTRACT

A device for supporting lateral flow of a liquid. The device includes a support layer having a fluid impervious material with a porated region to allow for penetration of liquid through the support layer, and a membrane layer of bonded microparticle adhered to the support. The device may be used in an apparatus for the detection of analytes in samples.

### CROSS-REFERENCE TO RELATED APPLICATION

This application claims the benefit of U.S. provisional application Ser. No. 63/436,280, filed Dec. 30, 2022, which is incorporated by reference in its entirety.

### FIELD

The disclosure is directed to devices for determining the presence of analytes in samples. In some embodiments, the devices are used in lateral flow immunoassays.

### BACKGROUND

Lateral flow immunoassays are useful for the determination of analytes in samples. However, challenges remain regarding the use of small sample sizes, including unprocessed whole blood samples. Accordingly, the inventors have developed the present devices that accommodate small sample sizes of all types.

### SUMMARY

In one aspect, the disclosure is directed to a device for supporting lateral flow of a liquid. The device includes a support layer that includes a fluid impervious material with a porated region having one or more pores, and a membrane layer adhered to the support layer and including a matrix of bonded microparticles. The matrix may include interstitial space between the bonded microparticles to allow lateral liquid flow through the membrane. The hydrophilic microparticle matrix may include glass microparticles, for example soda lime glass microparticles, or polyethylene microspheres.

In one embodiment, the matrix includes a water-insoluble binder that bonds the microparticles and adheres the microparticles to the support layer. The binder may be, for example, a polyurethane or a water emulsified latex.

In another embodiment, the membrane layer may include a sample application zone and a detection zone in fluid communication and laterally spaced from the sample application zone. The sample application zone may include a dried conjugate reagent that is solubilized by the sample upon addition of a liquid sample to the membrane layer an the detection zone may include an immobilized binding partner for the analyte.

In one aspect, the membrane of the device of the disclosure supports bilateral liquid flow. In addition, the device may be configured to accept a whole blood sample, for example, a small volume sample having less than 200 microliters. The sample may be undiluted whole blood, and the device, may include a filter laminated to a portion of the membrane layer including a sample application zone.

In another aspect, the disclosure is directed to a method of making the device for supporting lateral flow of a liquid. The method includes forming a slurry including the microparticles, a binder and a volatilizable solvent, applying the slurry to the support layer to provide the membrane layer, and allowing the slurry to dry to bond the microparticles to each other and to adhere the microparticles to the support layer.

In some embodiments, the slurry may include a surfactant, and the binder may include a polyurethane or a water emulsified latex. The method may further include laser ablating pores into the support layer.

The device of the disclosure may be inserted into an apparatus for detecting an analyte in a sample that includes the device and a housing. The housing may include a sealed reagent reservoir and may further include a piercing member to pierce the sealed reservoir and provide fluid communication between the reservoir and the membrane layer through a first porated region.

### BRIEF DESCRIPTION OF FIGURES

The accompanying drawings, which are included to provide a further understanding of the disclosure, are incorporated in and constitute a part of this specification, illustrate embodiments of the disclosure, and together with the detailed description serve to explain the principles of the disclosure. No attempt is made to show structural details of the disclosure in more detail than may be necessary for a fundamental understanding of the disclosure and various ways in which it may be practiced.

FIG. 1A and FIG. 1B respectively show a side view and a top view of a representation of the device according to the disclosure.

FIG. 2A and FIG. 2B respectively show an apparatus according the disclosure in an unactivated and activated configuration.

FIG. 2C shows a top view of an apparatus for conducting an immunoassay that incorporates the device according to the disclosure.

### DESCRIPTION

In various aspects, the disclosure provides for a device for supporting lateral flow of a liquid. Such devices are often used for processing samples for analysis of various substances (“analytes”) in liquid samples. As part of the process, a liquid sample suspected of containing an analyte is added to an area of the device for receiving the sample. The device allows for the lateral flow of the sample across one or more additional areas of the device so that the sample encounters the necessary reagents for an analysis method that allows for the determination of the presence and/or amount of the analyte in the sample.

The term “antigen,” as used herein, generally refers to a substance that is capable, under appropriate conditions, of reacting with an antibody specific for the antigen.

The term “antibody” as used herein, includes a polyclonal antibody, a monoclonal antibody, a single chain antibody (scFv), or an antigen binding fragment of an antibody. Antigen-binding fragments of antibodies are a portion of an intact antibody comprising the antigen binding site or variable region of an intact antibody, wherein the portion is free of the constant heavy chain domains of the Fc region of the intact antibody. Examples of antigen binding antibody fragments include Fab, Fab′, Fab′-SH, F(ab′)₂ and Fv fragments. An antibody can be any antibody class, including for example, IgG, IgM, IgA, IgD and IgE.

The term “analyte,” as used herein, generally refers to the substance, or set of substances in a sample that are detected and/or measured. As used herein, “analyte” generally refers to a molecule (e.g., antibody or antigen) that is present in a sample, such as a biological fluid, whose presence or concentration in the sample is intended to be determined and which binds to (i.e., forms a complex with) a binding partner (e.g., antigen or antibody). An analyte may be, for example, a protein, a glycoprotein, a saccharide, a polysaccharide, an amino acid, a substituted amino acid, a methylated amino acid, a hormone, an antibiotic, a nucleic acid, a metabolite, or a derivative of any of the foregoing.

The term “analyte analog,” as used herein, generally refers to a an analyte compound in which one or more individual atoms have been replaced with a different atom(s) or with a different functional group(s) that provide a means to join the analyte to another moiety, such as a label or solid matrix. For example, a means to join the analyte to another moiety may be a linker. An analog may compete with the analyte for a receptor. For example, the analyte analog can bind to an antibody in a manner similar to the analyte. Because covalent binding of the analyte to a matrix or another molecule is often accomplished through the use of an analyte analog, the disclosure herein of simply “the analyte” attached to or conjugated to a matrix or another molecule includes the use of an analyte analog to accomplish such covalent attachment or conjugation as would be readily understood by one of ordinary skill in the art of immunoassays.

The phrase “binding partner,” as that phrase is used herein, means a molecule that binds a second molecule with specificity. For example, the second molecule can be an antigen/antibody and the “binding partner” can be an antibody/antigen. These techniques are not limited to antibody-antigen binding partners as is generally known the art of lateral flow assays.

The term “animal” as used herein, generally refers to any animal, e.g., a human, or a non-human animal including companion animals, livestock, and animals in the wild.

The term “sample,” as used herein, generally refers to a sample fluid from a human or animal including, but not limited to whole blood, plasma, serum, spinal fluid, lymph fluid, abdominal fluid (ascites), the external sections of skin, respiratory, intestinal and genitourinary tracts, tears, saliva, feces and urine. Many such samples require processing prior to analysis, which may include, for example, separation, filtration, centrifugation, and the addition of stabilizers or diluents. Solid samples may be used to the extent that sample processing can render an analyte associated with the sample in solution. Samples include both raw samples (e.g., whole blood) and/or processed samples.

The term “blood sample,” as used herein, generally refers to any blood-derived fluid sample, including but not limited to whole blood, plasma, and serum. To provide serum for use in the methods of the disclosure, one or more serum samples are obtained from the animal subject. The serum samples can be, for example, obtained from the animal subject as blood samples, then separated to provide serum. In certain embodiments, the serum can be measured without separation from blood. As the person of skill in the art will appreciate, a single obtained sample can be divided or otherwise used to do both concentration measurements.

The term “immunoassay,” as used herein, generally refers to a test that employs antibody and antigen complexes to generate a measurable response. An “antibody:antigen complex” may be used interchangeably with the term “immunological complex.” Immunoassay is a technique for measuring the presence or concentration of a substance in a test sample, typically a solution, that frequently contains a complex mixture of substances.

Immunoassays, in general, include noncompetitive immunoassays, competitive immunoassays, homogeneous immunoassays, and heterogeneous immunoassays. Immunoassays that require separation of bound antibody:antigen complexes are generally referred to as “heterogeneous immunoassays,” and immunoassays that do not require separation of antibody:antigen complexes are generally referred to as “homogeneous immunoassays.” One of skill in the art would readily understand the various immunoassay formats.

The term “immunological complexes,” as used herein, generally refers to the complexes formed by the binding of antigen and antibody molecules. When one of either the antibody or antigen is labeled, the label is associated with the immune complex as a result of the binding between the antigen and antibody. Therefore, when the antibody is labeled, the label becomes associated with the antigen as a result of the binding. Similarly, when the antigen is labeled (e.g., an analyte analog having a label), the label becomes associated with the antibody as a result of the binding between the antigen and the antibody.

The term “label,” as used herein, refers to a detectable compound or composition, which can be conjugated to the analyte, a binding partner for the analyte, or another binding partner (e.g, antibody, analyte analog, or antigen) that participates in an immunoassay. The label may be detectable by itself (e.g., radioisotope labels, chemiluminescent dye, electrochemical labels, metal chelates, latex particles, or fluorescent labels) or, in the case of an enzymatic label, may catalyze chemical alteration of a substrate compound or composition which is detectable (e.g., enzymes such as horseradish peroxidase, alkaline phosphatase, and the like). The label employed in the current disclosure could be, but is not limited to: alkaline phosphatase; glucose-6-phosphate dehydrogenase (“G6PDH”), horse radish peroxidase (HRP), chemiluminescers such as isoluminol, fluorescers such as fluorescein and rhodamine compounds, and dyes. The utilization of a label produces a signal that may be detected by means such as detection of electromagnetic radiation or direct visualization, and that can optionally be measured.

The term “about,” as used herein means ±10%, preferably ±5%, more preferably, ±2%, and most preferably ±1%.

Turning now to the various aspects of the disclosure, the device of the disclosure includes a multilayer, generally flat sheet having an opaque support layer and a membrane layer. The support layer includes a fluid impervious material that may have one or more porated region(s) having one or more pore(s) to allow for penetration of liquid through the support layer. In some embodiments, the support layer is bright white or sufficiently white in order to maximize the visual contrast of the background and the label associated with the device as described herein.

The membrane layer is adhered to the support layer and includes a matrix of bonded microparticles. The microparticles may be bonded in a manner that provides for interstitial space between the microparticles to allow lateral liquid flow through the membrane. The amount of interstitial space depends on the size(s) of the bonded microparticles. The pores in the support layer allow fluid communication through the support layer and into the membrane layer.

The membrane layer may include a sample application zone and a detection zone in fluid communication with and laterally spaced from a sample application zone. Sample added to the sample application zone flows laterally through the membrane layer and through the detection zone. The detection zone includes one more reagents that are immobilized within the zone and interact with the analyte (or other components) in the sample in order to allow for the determination of the presence or amount of the analyte in the sample. In one aspect of the disclosure, a label that becomes immobilized within the detection zone as a result of the presence or absence of the analyte in the sample provides a detectable signal at the detection zone. Liquid reagents necessary for the determination of the analyte may be added to, and excess reagent may be removed from, the device through the porous regions of the support layer. In addition, the membrane layer may include a reagent zone that includes solubilizable reagents that are solubilized by the liquid sample or the liquid reagents and that contribute to the determination of the analyte in the sample.

Immunoassays using lateral flow devices are generally known. For instance, U.S. Pat. Nos. 5,750,333, 6,435,722, and 7,816,122 (which are incorporated herein in their entirety) describe various aspects of an apparatus that includes a test strip for determining an analyte in a sample and on-board reagents that are used in the detection steps. Such apparatuses include a test strip with a sample application zone, a detection zone and on-board reagents that participate in the detection of the analyte in the sample. Sample added to a test strip flows towards and through the detection zone, upon which a user “activates” the device to introduce the reagents onto the strip test and reverse the direction of the flow towards an absorbent pad in fluid communication with the test strip in order to absorb the excess liquid.

The device can be incorporated into a housing that supports the device, provides reservoirs for one or more on-board reagents, and provides the means to transfer the on-board reagents to the device, all of which allow for the determination of one or more analytes in a sample with minimal user interaction.

In one aspect, the disclosure is directed to a process for manufacturing the device as described herein. The process includes forming a slurry of the microparticles and a water insoluble binder, applying the slurry to the support layer, and allowing the slurry to dry to provide the membrane layer. The support layer can be rigid or flexible, and can provide structural stability to the membrane layer, which allows for ease of handling of the device and manufacturability of an apparatus.

Any suitable binder can be selected for properties associated with coatability of the support layer, the three dimensional porous structure formation upon drying, and a hydrophilic/hydrophobic balance. Examples of suitable water insoluble binders include polyurethane, polyvinylacetate, and water emulsified latex. The slurry includes a water based solvent or a water/alcohol based solvent with alcohol content ranging from 0 to 100%, a surfactant (for example a cationic, anionic, zwitterionic, or silicone based surfactant) to improve coated membrane wettability, and may also include a plasticizer (e.g., PEG 300) to improve coated membrane flexibility.

The microparticles forming the matrix of the membrane layer bond to each other and the support layer once the slurry dries upon volatilization of the solvent. Once the slurry dries, the binder can be substantially insoluble in an aqueous solution (for example, the sample) and hydrophilic to support the lateral flow of the sample across the matrix.

The device according to the disclosure provides a number of advantages over known test strips. For example, the support layer provides physical support to the membrane layer and enables the conveyance of the device into a continuous manufacturing process (e.g., reel to reel). The whiteness of the support layer may also increase the visual contrast of the background and the label in the detection zone to improve the visibility of the label. The device of the disclosure also advantageously allows for the use of small volume samples such as whole blood samples having volumes as low as about 100 microliters.

In various embodiments of the disclosure, the support layer is approximately 100-150 micrometers thickness and may be treated (primed) with a water insoluble polyurethane hydrogel/cellulose layer (about 10-30 μm thick) to increase the adhesion of the membrane layer. The support layer may be bright white or sufficiently white and non-transparent to enhance visualization of the label by the operator of the appropriate reading device.

In one embodiment, the microparticle/water insoluble binder slurry includes a surfactant (e.g. DOW 193C), microparticles ranging in size from 10-200 μm, and a water insoluble binder (i.e. polyurethane) in a 10/90 water/EtOH solvent or water emulsified (i.e. latex) binder in a water only solvent with a water soluble plasticizer (i.e. PEG 300). The microparticle/binder slurry is coated onto the primed support layer using a knife, a slot die coater or other common die cutting manufacturing techniques. The slurry is applied in a flat continuous coat to the primed support layer to a desired thickness, between about 125 μm and 1500 μm, depending on the flow properties desired. The porous structure of the membrane is created by the volatilization of the solvent from the slurry during the drying process. The binder serves to adhere the microparticles together and to adhere the microparticles to the support layer. The evaporated solvent leaves open the interstitial spaces between the microparticles. The microparticle coated support layer may be cut to a size appropriate to fit into a housing of a lateral flow apparatus.

Material useful for the support layer include rigid or semi-rigid materials that are compatible with the solvent and that provide suitable support for the membrane layer to allow for case of manufacturability as described above. In various embodiments, the support layer is water-insoluble and water-impermeable. In embodiments, the support layer is mechanically and thermally stable and scratch resistant. The support layer can be any suitable polymer that meets these criteria. Illustrative thin film materials that are suitable for a support layer include, but are not limited to, glass, polystyrene, polyesters, polycarbonates, cellulose derivatives (such as cellulose acetate), polyethylene terephthalate, and mixtures thereof. In one embodiment, the support layer is a polyester commercially available under the tradename Melinex® (commercially available from Tekra, a division of EIS, Inc. of Berlin, Wis.). In one embodiment, the support layer is a polyester commercially available under the tradename ESTAR™ (commercially available from the Eastman Kodak Company of Rochester, N.Y.).

The thickness of the support layer typically ranges from about 15 μm to about 200 μm, preferably about 50 μm to about 150 μm, and more preferably about 70 μm to about 130 μm. In one embodiment, the thickness of the support layer is about 125 μm. In one embodiment, the thickness of the support layer is about 75 μm.

The fluid impervious support layer can permit fluid to pass through in a specific region of the membrane by creating spatially defined features such as pores (in any form including slits, cuts, holes, etc.) in the support layer using, for example, laser ablation. For example, a 3-axis CO₂ laser is used for the ablation of the support layer without damaging the underlying membrane layer. The pores can range in size from about 10 microns to 5 mm and may vary in numbers between 5 to 1000, depending on the size of the support layer and to the extent that the pores do not substantially impair the rigidity of semi-rigidity of the support layer.

Microparticles useful for the various aspects of the disclosure include, for example, glass, e.g. soda lime glass, or polymeric, e.g. polyethylene, particles. Other materials are also useful that are compatible with the binder and solvent used in the manufacturing process. Such materials include, but are not limited to latex, polystyrene, silica, agarose, ceramics, polyacrylamides, polymethyl methacrylates, carboxylate modified latex, melamine, and Sepharose. In particular, useful commercially available materials include carboxylate modified latex, cyanogen bromide activated Sepharose beads, fused silica particles, isothiocyanate glass, polystyrene, and carboxylate monodisperse microsphere microparticles. In example embodiments, he particles vary in size from about 10 microns to about 200 microns, for example about 10, 20, 30, 40 50, 60, 70, 80 90, 100, 110, 120, 130, 140, 150, 160, 170, 180, 190 or 200 microns. The particles may be homogenous or heterogeneous in material and size as long as they are compatible with the binder, solvent, and each other. In some embodiments the particles are between about 30 microns and 60 microns.

Use of microparticles of varying size allows for the adjustment of the speed of lateral flow through the device by changing the interstitial spaces between the particles of the membrane. For instance, a coated membrane composed of larger sized microparticles has faster sample flow rate and assay time-to-result than that of smaller sized microparticles. Applicants determined that the membrane of the disclosure advantageously allows for the use of small volume samples such as whole blood samples having volumes as low as about 100 microliters, for example about 125 microliters, 150 microliters, 175 microliters, or 200 microliter.

The membrane layer of the device can be laminated with other membranes, for example using single sided pressure sensitive adhesive tapes or other adhesives positioned to avoid impairment of fluid permeation through the filter. For example, Whatman VF2 whole blood filter which may be impregnated with other chemicals (e.g., NaCl) or biological excipients (e.g, anti-RBC, lectin) to permit effective capturing of red blood cells in undiluted whole blood samples. In example embodiments, the filter layer includes a porous filter (e.g. Whatman VF2 filter) that has been soaked in a solution of sodium chloride and allowed to dry. For example, the solution contains sodium chloride at about 0.25 to about 0.3 molar, such as 0.25 M, 0.5 M, 1.0 M, 1.5 M, 2.0 M, 2.5 M or 3.0 M. A sample added to the filter solubilizes the sodium chloride and enhances the capture of red blood cells on the filter. In another example embodiment, the filter can be similarly impregnated with lectin.

Once manufacturing of the device is complete, the membrane may be functionalized with reagents appropriate for conducting an assay. Accordingly, the binder-coated particles should be capable of attachment to other substances such as derivatives, linker molecules or proteins. The capability of the membrane to be attached to other substances can result from the binder and particle materials as well as from any surface modifications or functional groups added to the membrane. The membrane can be functionalized or be capable of becoming functionalized in order to covalently or non-covalently attach proteins, antibodies, linker molecules or derivatives as described herein. Suitable functional groups include, for example, amine, biotin, streptavidin, avidin, protein A, sulfhydryl, hydroxyl and carboxyl.

In addition, one or more reagents useful in immunoassays can be non-diffusively bound in a reagent zone of the device such that the liquid sample can solubilize the reagent(s) as the sample liquid passes through the reagent zone and towards the detection zone. The solubilized reagents can participate in the immunoassay conducted on the device as is known in the art. For example, the reagent in the reagent zone may be labeled anti-analyte antibody conjugate that binds with analyte in the sample as the sample migrates past and towards the detection zone. The complex of the analyte the conjugate then binds to another binding partner for the analyte that is immobilized in the detection zone. Another example includes a labeled analyte analog that binds an anti-analyte antibody immobilized in the detection zone such that the labeled analyte analog competes with analyte the sample for binding to the antibody in the detection zone.

When used in an apparatus for conducting an analysis of a sample, areas of the device may be designated as a sample application zone, a reagent zone, and a detection zone. The device supports the lateral flow of the sample that is added to the sample application zone towards the reagent zone and the detection zone. In some embodiments as further described herein the direction of the flow is reversed after the sample passes the detection zone to wash unbound materials from the detection zone. In addition, if the sample has been processed, prior to its application to the device, by the addition of reagents that would otherwise be diffusively bound in the reagent zone, the reagent zone may be absent from the device.

In various embodiments, depending on whether the assay is conducted as immunological sandwich assay or a competitive assay, for example, the detection zone may include a binding partner to capture the analyte or an analyte analog that would capture a binding partner for the analyte. In a sandwich assay, the analyte may be captured in the detection zone by a binding partner for the analyte (e.g., an antibody) that is non-diffusively bound in the detection zone, and then sandwiched with a second binding partner that includes a detectable label. The binding partner/label complex is known as “conjugate” reagent that, depending on the label, may provide a visual signal in the detection zone with or without the addition of other reagents to the detection zone. The conjugate may be added to the sample or may be dried and diffusively bound in the reagent zone of the device. If the conjugate is diffusively bound, it is solubilized by the sample and moves with the liquid flow toward the detection zone.

In a competitive assay, an analog of the analyte is bound to the detection zone and conjugate that binds to analyte in the sample is prevented from binding to the analyte analog in the detection zone. The absence of signal is an indication of the presence of the analyte in the sample.

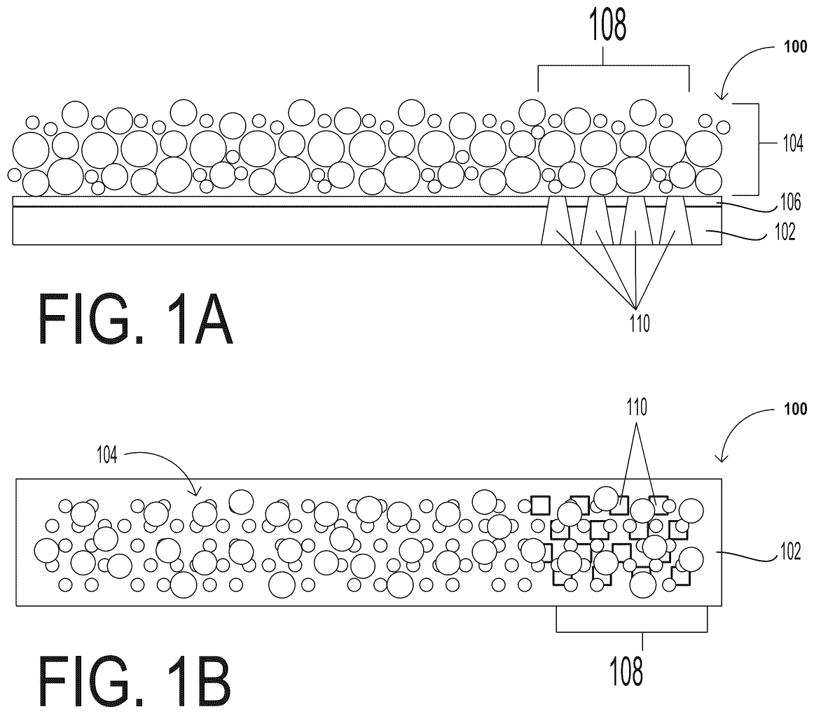

An example of a device according the disclosure is shown in FIGS. 1A-1B. The device 100 is configured to support lateral flow of a liquid. The device 100 includes a support layer 102 and a membrane layer 104 adhered to the support layer 102. In some embodiments, device 100 could further include a primer layer, such as the primer layer 106 shown in FIG. 1A, such that the primer layer 106 is between the support layer 102 and the membrane layer 104 in order to aid the adhesion of the membrane layer 104 to the support layer 102.

The support layer 102 includes a fluid impervious material that prevents fluid flow through the support layer. However, the support layer 102 (and primer layer 106, if present) may include a porated region 108 that includes one or more pores 110 that allow for penetration of liquid through the support layer 102 (and primer layer 106). In some embodiments, the support layer 102 (and primer layer 106) may include more than one porated region 108 with pores 110 that allow for penetration of liquid through the support (and primer) layers.

In some embodiments, the membrane layer 104 may include a sample application zone and a detection zone that is in fluid communication with, but laterally spaced apart from, the sample application zone. Once a sample is applied to the sample application zone, it will migrate by lateral flow towards the detection zone. In some embodiments, the sample application zone further includes a dried conjugate reagent that is solubilized by a sample upon addition of a liquid sample to the membrane layer 104. In other embodiments, the dried conjugate reagent may be positioned in a reagent zone region that is laterally spaced between the sample application zone and the detection zone such that the fluid flow from the sample application zone to the detection zone solubilizes the reagents in the reagent zone and transports them to the detection zone. In some embodiments, the detection zone includes an immobilized binding partner for an analyte or analog of the analyte as known the art of lateral flow immunoassays.

In some embodiments, the device 100 supports fluid flow in all directions. In example embodiments, a device according to the disclosure is in the form of a test strip such that liquid applied at one end of the strip will saturate the width of the strip and migrate towards the opposite end. Lateral flow is directed by fluid pressure, which can be reversed by fluid added in added at location on the device that results in an opposing direction of flow. As such, liquid can move in one direction across the device 100 or in an opposite direction across the device 100. In some embodiments, the device 100 further includes a filter layer (not shown) that is laminated to a portion of the membrane layer 104 with single sided pressure sensitive adhesive tapes or other adhesive positioned to avoid impairment of fluid permeation through the filter. The filter layer may remove components of the sample (e.g., red blood cells) and may include reagents that are added to the sample prior to the sample contacting the membrane layer 104.

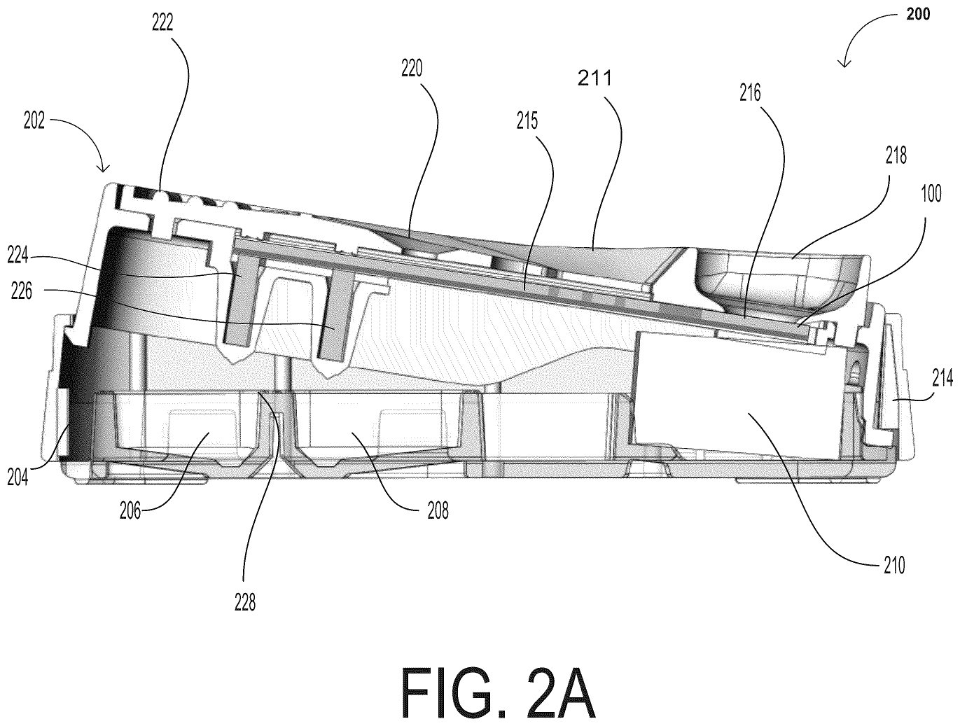

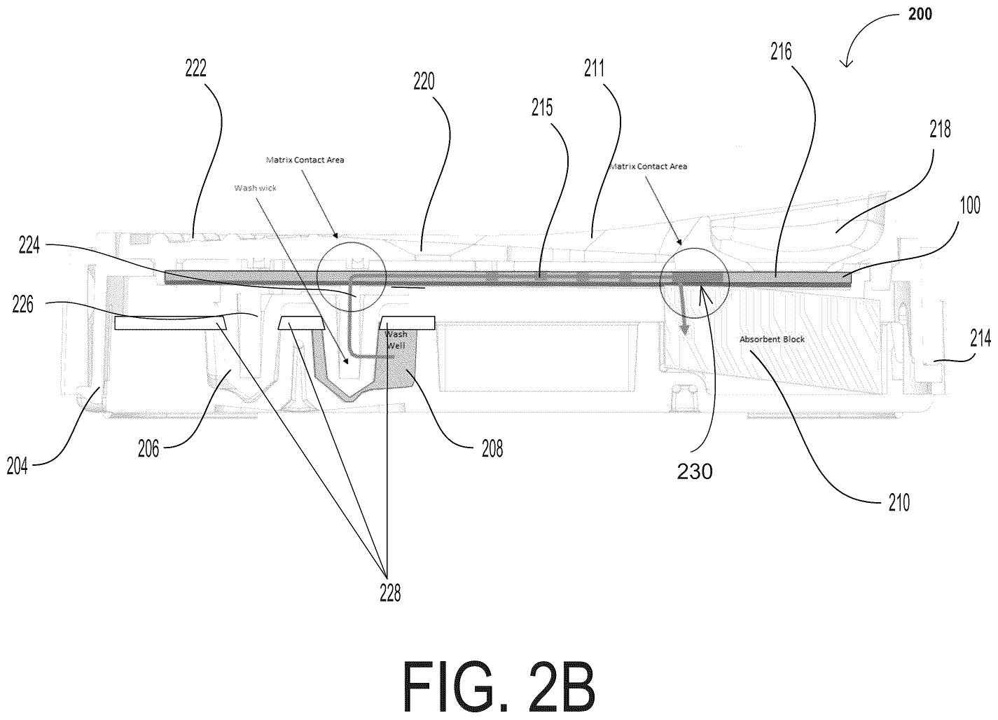

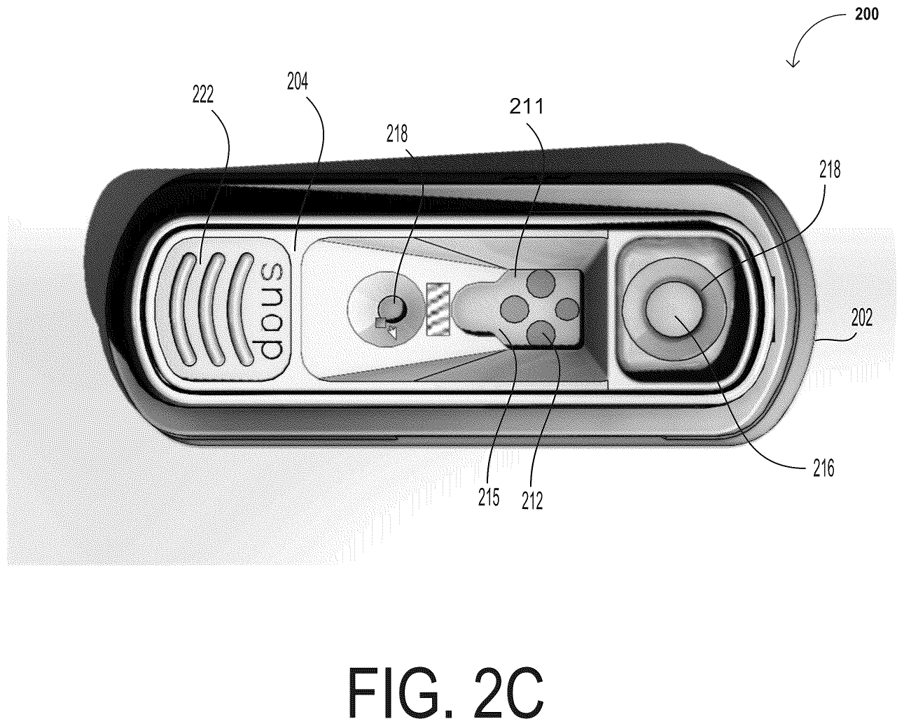

Referring now to FIGS. 2A and 2B, an example of the apparatus 200 is shown in both the un-activated (FIG. 2A) and the activated (FIG. 2B) configuration. The apparatus includes a two-part housing having an upper housing portion 202 that secures the device 100. A lower housing portion 204 secures reagent containers 206 and 208 and an absorbent pad 210. The upper housing portion 202 provides a window 211 that allows an operator to visually monitor assay results at a detection zone 215 of the membrane layer 104 through the window 211. Accordingly, the device is secured in the housing such that the membrane layer the top layer such that an operator can visually view the membrane layer through the window 211. In FIG. 2C, an immobilized binding reagent 212 is represented in a detection zone 215 as dots that are viewable through the window. As alternative to dots, any shape can be used, including + or − signs, dashes, or any other shape that can provide a visual signal to the operator. The relative sizes of components of the device 100 and apparatus 200 can vary and the proportions depicted in the figures herein are for illustrative purposes only.

The upper housing portion 202 and the lower housing portion 204 are pivotably disposed with respect to each other by means of a hinge 214. The pivotal connection initially holds the two portions of the housing in a pre-activated configuration prior to the addition of a sample liquid to a sample application zone 216 of the device. The upper housing portion 202 may include a sample application cup 218 or similar structure to assist the operator to apply the sample to the device in the correct location in a sample application zone 216. An aperture 220 in the upper housing portion 202 that allows the operator to visually determine that the sample, once applied to the sample application zone 216, has sufficiently migrated along the membrane from the sample application zone and past an immobilized binding reagent 212 in the detection zone 215. The membrane layer 104 may include a reagent that changes color in the area of the aperture that allows the operator to clearly determine that the liquid has sufficiently migrated to the location of the aperture.

The membrane layer may include a reagent zone (not shown) that includes a reagent that is diffusively applied to the reagent zone such that it can be solubilized by the liquid sample and participate in the assay. In one embodiment, the reagent zone is laterally spaced between the detection zone 215 and the sample application zone 216. In another embodiment, the detection zone overlaps or is congruent with the sample application zone. In various embodiments, the reagent in the reagent zone may be labeled anti-analyte antibody conjugate that binds with analyte in the sample as the sample migrates towards the detection zone. The complex of the analyte the conjugate then binds to another binding partner for the analyte that is immobilized in the detection zone. Another example of a reagent in the reagent zone includes a labeled analyte analog that is diffusively bound in the reagent zone and that binds an anti-analyte antibody in the detection zone in competition with analyte in the sample.

Once liquid sample has reached the location of the aperture 220, the operator may activate the apparatus by pressing at a location 222 on the upper housing portion 202 to cause the upper housing portion 202 to pivot on hinge 214 and to nest and lock into lower housing portion 204. Activation causes the device 100 to contact the absorbent pad 210. Activation also releases the liquid reagents from the sealed containers 206 and 208 enabling flow of the liquid reagents into the membrane through spikes 224 and 226, which include a rigid component for piercing a seal 228 on the containers 206 and 208 and a wick-like material that allows for liquid to migrate from the containers to the membrane. Activation of the apparatus locks the apparatus with pressure sufficient to (a) compress the absorbent pad 210 such that a contact portion 230 of the pad can contact the membrane layer 104 through the pores of the support layer 102 to provide fluid communication between the membrane layer and the pad, and (b) compress a portion of the wicking material associated with the spikes such that the wicking material contacts the membrane layer 104 through a pores (not shown) in the support layer at the location of the spikes that provide fluid communication through the support layer between the spikes and the membrane layer.

In the device illustrated in 2A and 2B, the liquid reagent containers 206 and 208 contain a wash reagent and a detector reagent. The wash reagent is stored reagent container 208 and is delivered by the wash reagent delivery spike 226 to the membrane layer 104. The wash reagent reverses the flow of the liquid towards the absorbent pad 210 and transports unbound sample and unbound conjugate along the membrane and away from the detection zone 215 and into the absorbent pad 210.

The detector reagent containing the conjugate is stored in the reagent container 206 and is delivered by spike 224 through the pores 110 in the support layer 102 and to the membrane layer 104. The detector reagent facilitates analyte detection. Sequential utilization of the two reagents, i.e., wash reagent followed by detector reagent, is accomplished by delivering the wash reagent closer to the absorbent pad 210 than the detector reagent. Fluid flow toward the absorbent pad 210 after activation causes the wash reagent be pulled into the membrane by capillary force. Once a volume of the wash reagent been absorbed into the membrane layer 104, displacing unbound sample and unbound conjugate reagent, the detector reagent is delivered into the membrane by capillary force. The detector reagent displaces the wash reagent in the direction of the absorbent pad 210. The detector reagent may include a substrate that provides a detectable signal in the presence of an immune complex in the detection zone. In some embodiments where the label does not require a substrate, the detector reagent and reagent container 206 may not be necessary.

What is claimed is:

1. A device for supporting lateral flow of a liquid, comprising
a support layer comprising a fluid impervious material having a porated region with one or more pores, and
a membrane layer adhered to the support layer and comprising a matrix of bonded microparticles.

2. The device of claim 1, where the matrix comprises interstitial space between the bonded microparticles to allow lateral liquid flow through the membrane.

3. The device of claim 1, wherein the matrix comprises glass microparticles or polyethylene microspheres.

4. The device of claim 3, wherein the glass microparticles are soda lime glass microparticles.

5. The device of claim 1, wherein the matrix comprises a water-insoluble binder that bonds the microparticles and adheres the microparticles to the support layer.

6. The device of claim 5, wherein the binder comprises a polyurethane or water emulsified latex.

7. The device of claim 1, wherein the membrane layer comprises a sample application zone and a detection zone in fluid communication and laterally spaced from the sample application zone.

8. The device of claim 7, wherein the sample application zone further comprises a dried conjugate reagent that is solubilized by the sample upon addition of a liquid sample to the membrane layer.

9. The device of claim 7, wherein the detection zone comprising an immobilized binding partner for an analyte.

10. The device of claim 1, wherein the membrane supports bilateral liquid flow.

11. The device of claim 1, wherein the device is configured to accept a whole blood sample.

12. The device of claim 1, wherein a sample volume is less than 200 microliters.

13. The device of claim 1, further comprising a sample of undiluted whole blood.

14. The device of claim 1, further comprising filter laminated to a portion of the membrane layer comprising a sample application zone.

15. A method of making the device of claim 1, comprising
forming a slurry comprising the microparticles, a binder and a volatilizable solvent;
applying the slurry to the support layer to provide the membrane layer; and
allowing the slurry to dry to bond the microparticles to each other and to adhere the microparticles to the support layer.

16. The method of claim 15, wherein the slurry further comprises a surfactant.

17. The method of claim 15, wherein the binder comprises a polyurethane or a water emulsified latex.

18. The method of claim 15, wherein the solvent is 10% water and 90% alcohol.

19. The method of claim 15, further comprising priming the support layer prior to the applying the slurry.

20. The method of claim 19, wherein the priming comprises applying a water insoluble primer comprising at least one of a polyurethane hydrogel and cellulose to the support layer.

21. The method of claim 20, further comprising laser ablating pores into the support layer.

22. An apparatus for detecting an analyte in a sample, comprising:
the device of claim 1 and a housing.

23. The apparatus of claim 22, further comprising a sealed reagent reservoir and a piercing member to pierce the sealed reagent reservoir and provide fluid communication between the reservoir and the membrane layer through a first porated region.

24. The apparatus of claim 23, further comprising an absorbent block in fluid communication with the membrane through a second porated region.

25. The apparatus of claim 24, wherein a detection zone is located between the first and second porated regions.
